(19) United States
(12) Patent Application Publication
(10) Pub. No.: US 2010/0021471 A1
(43) Pub. Date: Jan. 28, 2010

(54) CARBON NANOTUBE-BASED DRUG DELIVERY SYSTEMS AND METHODS OF MAKING SAME

(75) Inventors: Jingyi Chen, St. Louis, MO (US); Stanislaus S. Wong, Stony Brook, NY (US); Iwao Ojima, Port Jefferson, NY (US)

Correspondence Address: BROOKHAVEN SCIENCE ASSOCIATES/;BROOKHAVEN NATIONAL LABORATORY, BLDG. 490C - P.O. BOX 5000, UPTON, NY, 11973, US

(73) Assignee: Brookhaven Science Associates, LLC, Upton, NY (US)

(21) Appl. No.: 12/179,887

(22) Filed: Jul. 25, 2008

(51) Int. Cl. A61K 39/395; A61K 31/415; A61K 31/495; A61K 31/715; A61K 31/337; A61P 35/00

(52) U.S. Cl. 424/141.1; 514/393; 514/249; 514/54; 514/449

(57) ABSTRACT

The present invention includes a conjugates comprising a carbon nanotube with at least one covalently attached recognition module, and at least one covalently attached pharmaceutical compound or a precursor of the pharmaceutical compound,

wherein the pharmaceutical compound, or precursor of the pharmaceutical compound, is attached to the carbon nanotube by a linker moiety.

This application was supported by grants from the National Cancer Institute (CA 103314) and the National Science Foundation (CAREER DMR-0348239). The application was also supported by the U.S. Department of Energy Office of Basic Energy Sciences under Contract DE-AC-02-98CH10886. The Government has certain rights in this application.

### BACKGROUND OF THE INVENTION

A number of approaches to the functionalization of carbon nanotubes (CNTs) with biomolecules on their external surface have been reported for potential applications to drug delivery (Lacerda et al., (2006) Adv. Drug Delivery Rev. 58: 1460-1470; Klumpp et al., (2006) Biochim. Biophys. Acta 1758: 404-412.) It has been shown that CNTs serve as a highly efficient vehicle to transport a wide range of molecules across membranes into living cells. CNTs, whose diameter range is approximately the same size as that of ion channels and gap junctions, possess a high aspect ratio and this allows for efficient permeation of transported drug molecules into tissues and cell membranes (Porter et al., (2007) Nature Nanotech. 2: 713-717). Moreover, the intrinsic stability and structural flexibility of CNTs may prolong the circulation time as well as bioavailability of transported drug molecules. As an example of their versatility, single-walled carbon nanotubes (SWNT) noncovalently bound to proteins or genes mediated by phospholipids were internalized into cells through endocytosis (Gao et al., (2006) ChemBioChem 7: 239-242, Kam et al., (2005) J. Am. Chem. Soc. 127: 6021-6026; Kam et al. (2006) Angew. Chem., Intl. Ed. 45: 577-581.) SWNTs can be covalently functionalized with small molecules linked to the carboxylic acid sites localized at their ends, defect sites, or sidewalls (Pastorin et al. (2006) Chem. Commun.: 1182-1184). Thus, amino acids, oligopeptides, and antibiotics have been transported into different types of cells via appropriately functionalized SWNTs (Georgakilas et al., (2002) Chem. Commun.: 3050-3051; Pantarotto et al. (2003) J. Am. Chem. Soc. 125: 6160-6164, Wu et al., (2005) Angew. Chem., Intl. Ed. 44: 6358-6362; Kostarelos et al., (2007) Nature Nanotech. 2: 108-113.) More recently, multiple cytotoxic platinum(IV) complex units were conjugated to SWNTs for delivering those anticancer drugs to tumor cells, mediated by phospholipids, wherein the active platinum(II) species (cis-platin) was released upon reduction at a low pH environment within the cancer cells (Feazell et al., (2007) J. Am. Chem. Soc. 129: 8438-8439).

However, these prior art methods do not allow for targeted therapy. Thus, these prior art methods can lead to systemic toxicity which is the cause of undesirable side effects in the conventional chemotherapy.

### SUMMARY OF THE INVENTION

In one embodiment, the present invention includes conjugates comprising a carbon nanotube with at least one covalently attached recognition module, and at least one covalently attached pharmaceutical compound or a precursor of the pharmaceutical compound. The pharmaceutical compound, or precursor of the pharmaceutical compound, is attached to the carbon nanotube by a linker moiety. Preferably, the linker moiety is a disulfide-containing linker.

Preferably, the recognition module is primarily attached to an end of the carbon nanotube; and the pharmaceutical compound, or a precursor of the pharmaceutical compound, is primarily attached to the sidewall of the carbon nanotube.

The recognition module is typically a protein, peptide, a polyunsaturated fatty acid, a saccharide, a lectin, an aptamer, a glycosaminoglycan, or a vitamin. For example, the recognition module can be biotin or folic acid. The recognition module can also be a monoclonal antibody or hyaluronic acid.

The pharmaceutical compound, or the precursor thereof, can be an antitumor drug, an antiangiogenic drug, a multi-drug reversal agent, an anti-inflammatory drug, an antibiotic, an antibacterial agent, an antiparasitic drug or an analgesic. For example, the pharmaceutical compound can be paclitaxel or a taxoid.

Preferred examples of taxoids include docetaxel, SB-T-101131, SB-T-1102, SB-T-1103, SB-T-11033, SB-T-1104, SB-T-1212, SB-T-1213, SB-T-121303, SB-T-1214, SB-T-1216, SB-T-1217, SB-T-12851, SB-T-12852, SB-T-12853, SB-T-12854 or SB-T-1250.

In another embodiment, the present invention includes methods of making a conjugate comprising a carbon nanotube with at least one covalently attached recognition module, and at least one covalently attached pharmaceutical compound or a precursor of a pharmaceutical compound.

The method comprises consecutively contacting a plurality of oxidized carbon nanotubes with (i) a first bifunctional amine having a first protecting group and (ii) a second bifunctional amine having a second protecting group to yield a plurality of carbon nanotubes with the first bifunctional amine primarily attached to the sidewall and the second bifunctional amine primarily attached to the end/defect sites. The first protecting group is replaced with a linker attached to the pharmaceutical compound or the precursor of a pharmaceutical compound, and the second protecting group is replaced with the recognition module. The replacements occur consecutively in either order to yield the conjugate.

In one embodiment, the first bifunctional amine is attached to the sidewall by 1,3-dipolar cycloaddition of azomethine ylide generated by condensation of an amino acid and an aldehyde. In another embodiment, the first bifunctional amine is attached to the sidewall by [2+1]cycloaddition of nitrenes.

In one embodiment, the second bifunctional amine is attached to the end/defect sites of the nanotubes by reacting the nanotubes with N-(4-aminobutyl)-phthalimide. In another embodiment, the second bifunctional amine is attached to the end/defect sites of the nanotubes by reacting the nanotubes with a 9-fluorenylmethyloxy-carbonyl (FMOC) group.

Examples of protecting groups include a carbobenzyloxy group; a tert-butyloxy-carbonyl group; a 9-fluorenylmethyloxycarbonyl group; a benzyl group; and a p-methoxyphenyl group. An example of a bifunctional amine is N-(2-N-protecting group-ethyl)glycine.

The present invention has several advantages over the prior art methods. The drug conjugates provide targeted therapy. Additionally, they are stable and innocuous in blood circulation, yet the conjugates are designed to activate their cytotoxic drug warhead by chemical or biochemical transformation inside cells.

For example, SWNT-anticancer drug conjugates are equipped with tumor-targeting ligand units that can recognize the cancer specific receptors on the cell surface and induce receptor-mediated endocytosis. This tumor-targeting strategy, exploiting cancer specific biomarkers and efficient internalization, minimizes systemic toxicity, which is the cause of undesirable side effects in the conventional chemotherapy.

### BRIEF DESCRIPTION OF DRAWINGS

FIG. 1. TEM images of HiPco SWNTs: (A) pristine; (B) acid oxidized SWNTs, (C) AFM image of the acid oxidized SWNTs, and (D) ATR-IR spectrum of acid oxidized SWNTs. [Note: the peak at 2349 cm⁻¹ is attributed to the asymmetric stretch mode of the CO₂ molecules in the atmosphere.]

FIG. 2. UV-visible spectra of SWNT and its conjugates: (A) acid oxidized SWNTs; (B) taxoid-fluorescein conjugates; and (C) biotin-SWNT-taxoid-fluorescein conjugate 3 and the blow-up spectrum in the inset showing the absorption peak of the conjugate 3 at the region between 400-600 nm.

FIG. 3. (A) UV-visible spectra of the conjugates 1 and 2. (B) Photographs of vials containing pristine SWNT 0, SWNT-FITC 1, biotin-SWNT-FITC 2 and biotin-SWNT-taxoid-fluorescein 3 in CH₂Cl₂.

FIG. 4. CFM images of L1210FR cells after incubation with SWNT-FITC (1) (A) and biotin-SWNT-FITC (2) (B) at the final concentration of 10 μg/mL at 37° C. for 2 h. (C) Comparison of fluorescence intensities of L1210FR cells by flow cytometry upon treatment with pristine SWNTs (0) (purple), conjugate 1 (blue), and conjugate 2 (red) at the final concentration of 10 μg/mL in each case. Background, i.e., data for untreated cells, is plotted in black.

FIG. 5. CFM images and the flow cytometry analysis of L1210FR cells after incubation with SWNT-FITC 1 at the final concentration of 10 μg/mL with different condition for 3 h: (A) at 37° C. for 3 h: (B) at 4° C.; and (C) at 37° C. with the presence of 0.05% NaN₃. (D) CFM images and flow cytometry data of L1210FR cells after treatment with oxidized SWNT at the same concentration at 37° C. for 3 h as the control experiment. All the CFM images and flow cytometry data are taken at the same condition.

FIG. 6. CFM images and the flow cytometry analysis of L1210FR cells after incubation with biotin-SWNT-FITC 2 at the final concentration of 10 μg/mL with different condition for 3 h: (A) at 37° C. for 2 h; (B) at 4° C.; (C) at 37° C. with the presence of 0.05% NaN₃; (D) at 37° C. after pretreatment with excess biotin. All the CFM images and flow cytometry data are taken at the same condition.

FIG. 7. CFM images of L1210FR cells treated with biotin-CNT-taxoid-fluorescein (3) incubated before (A) and after (B) the addition of GSH-ethyl ester. The image (B) clearly shows the fluorescent microtubule networks in the living cells generated by the binding of the fluorescent taxoid, SB-T-1214-fluorescein, after the cleavage of the disulfide bond in the linker by GSH or GSH-ethyl ester.

FIG. 8. CFM images and the flow cytometry analysis of different cell types after incubation with biotin-SWNT-taxoid conjugate 3 at the final concentration of 50 μg/mL at 37° C. for 3 h: (A) L1210LR that is over-expressed biotin receptors; (B) L1210; and (C) W138 noncancerous human embryo fibroblast cells. All the CFM images and flow cytometry data are taken at the same condition.

FIG. 9. Results of MTT cytotoxicity assay of biotin-SWNT-taxoid conjugate 3 in the presence of different cell lines: (A) L1210FR; (B) L1210; and (C) W138 human noncancerous cell line.

FIG. 10. Schematic illustration of three key steps involved in the tumor-targeting drug delivery of biotin-SWNT-taxoid conjugate 3: (1) internalization of the whole conjugate via receptor-mediated endocytosis; (2) drug release through cleavage of the disulfide linker moiety by intracellular thiol, e.g., GSH; (3) binding of the free taxoid molecules to tubulins/microtubules, forming stabilized microtubules that block cell mitosis and trigger apoptosis. [Note: Since each taxoid molecule is fluorescently labeled with fluorescein, the internalized conjugate 3 in the cytoplasm and the taxoid-bound microtubules are fluorescent.]

FIG. 11. (A) Structures of three functionalized SWNT-based conjugate drug delivery probes: SWNT-FITC 1, biotin-SWNT-FITC 2, and biotin-SWNT-linker-taxoid-fluorescein 3.

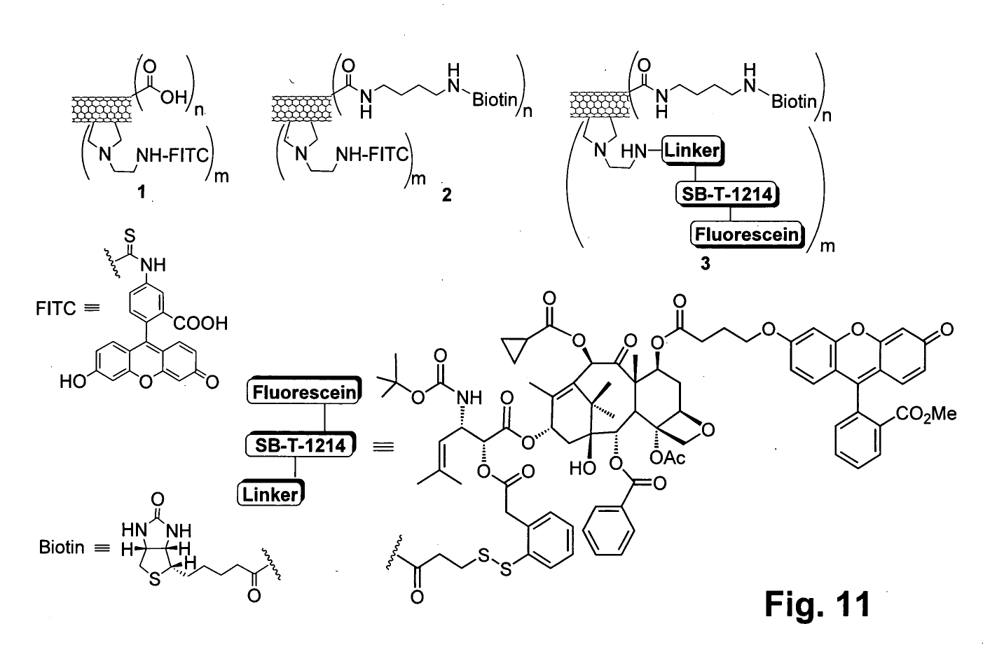

FIG. 12. Synthesis of conjugate 3 bearing a fluorescent taxoid as warhead.

FIG. 13. ¹H NMR spectrum of SuO-linker-taxoid (SB-T-1214)-fluorescein (13).

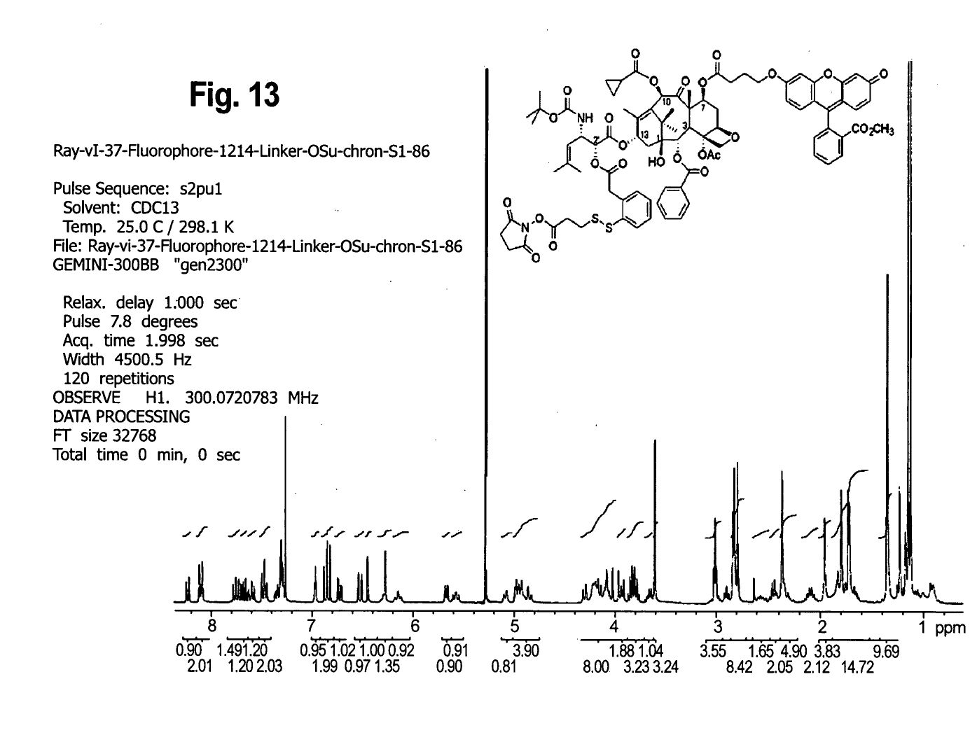

FIG. 14. The histogram of length (A) and height (B) measurements on 50 carbon nanotubes that are recorded by AFM.

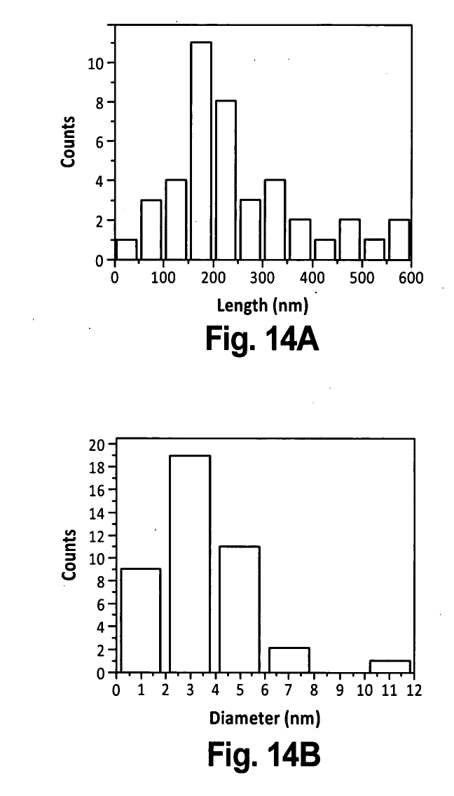

FIG. 15. ATR-IR spectra of biotin-SWNT-FITC conjugate 2.

FIG. 16. The photograph of SWNT conjugates at the concentration of 50 μg/mL in cell culture medium: (left) SWNT-FITC conjugate 1 and (right) biotin-SWNT-taxoid-fluorescein conjugate 3. The arrow indicated that some of nanotubes precipitated from the medium.

FIG. 17. The CFM images of an individual L1210FR cell staining with conjugate 3 in green (A), α-tubilin antibody in red (B); (C) phase contrast image of the cell, and (D) overlay of the image A, B and C. The microtubule network in the cell were clearly seen in the images. It is suggested that the taxoid molecules were released from the conjugates and bound to the microtubules.

FIG. 18. Preparation of oxidized SWNT 4.

FIG. 19. Synthesis of SWNT-FITC conjugate 1.

FIG. 20. Synthesis of biotin-SWNT-FITC conjugate 2.

FIG. 21. Synthesis of biotin-SWNT-linker-taxoid (SB-T-1214)-fluorescein conjugate 3.

### DETAILED DESCRIPTION OF THE INVENTION

The present invention provides drug delivery systems in the form of carbon nanotube conjugates and methods of making such conjugates.

Throughout this specification, there are ranges defined by upper and lower boundaries. Each lower boundary can be combined with each upper boundary to define a range. The lower and upper boundaries should each be taken as a separate element.

### Conjugates

A conjugate of the present invention comprises a carbon nanotube with at least one attached recognition module and at least one attached pharmaceutical compound or precursor of a pharmaceutical compound. (Hereinafter, a pharmaceutical compound and/or precursor of a pharmaceutical compound are collectively referred to as a “pharmaceutical.”)

The carbon nanotube of the conjugates comprises graphene in cylindrical form. The nanotube preferably has open ends. Alternatively, the nanotube can have one or two hemispherical caps on its ends. In addition to hexagonal carbon rings, the nanotube can comprise pentagonal rings of carbon. The carbon nanotube can be semi-conducting or metallic.

The carbon nanotube can be either a single-walled nanotube (SWNT) or a multi-walled nanotube (MWNT). A SWNT comprises only one nanotube. A MWNT comprises more than one nanotube each having a different diameter. Thus, the smallest diameter tube is encapsulated by a larger diameter tube, which in turn, is encapsulated by another larger diameter nanotube. A MWNT comprises, for example, up to about fifty nanotube shells. Preferably, the MWNT nanotube is a double-walled nanotube (DWNT).

SWNTs typically have a diameter of about 0.7 to about 2.5 nm, and a length of up to about one mm. MWNTs typically have a diameter of about 3 to about 30 nm, and a length of up to about one mm.

The recognition modules of the present invention are preferably covalently attached to the carbon nanotube. In a preferred embodiment, the recognition modules are primarily attached to the ends of a carbon nanotube.

In a preferred embodiment, the pharmaceuticals are primarily attached to the sidewalls of the nanotube. The quantity of pharmaceuticals that are attached to the sidewall of a carbon nanotube can be expressed by defining the percentage of carbon atoms which have a pharmaceutical attached. The percentage can be expressed as a percentage range. Preferably, the range of the quantity of sidewall carbon atoms that have a pharmaceutical attached has a lower boundary of approximately 3%. Examples of other lower boundaries include approximately 5%, 8%, 10% and 12%. Preferably, the range of the quantity of sidewall carbon atoms that have a pharmaceutical attached has an upper boundary of approximately 30%. Examples of other upper boundaries include approximately 16%, 20% and 25%. An example of a range is about 5 to 20%.

The ratio of a pharmaceutical to a recognition module is any ratio which provides a sufficient level of delivery of the pharmaceutical. Examples of ratios include from about 10:1 to about 1:10, more typically from about 5:1 to about 2:1, most typically about 3:1.

### Recognition Module

A recognition module of the present invention is a moiety that selectively binds to a molecule on the surface of a cell. Examples of a recognition module include proteins, peptides, lectins, saccharides, glycosaminoglycans (e.g., hyaluronic acid), polyunsaturated fatty acids, vitamins. and DNA and RNA oligomers (e.g., aptamers). Aptamers are short strands of DNA or RNA containing 15-35 nucleotides at 5-15 KDa.

Some types of molecules on the surface of a cell that may be targeted by a recognition module include receptors; glycoproteins; oligosaccharides; lectins; adhesion molecules; proteoglycams; integrins; immunoglobulins; major histocompatibility complex, e.g., human leukocyte antigen; and glycoproteins. Some examples of receptors include tyrosine kinase receptors, such as vascular endothelial growth factor (VEGF) receptor, and epidermal growth factor (EGF) receptors, e.g., HER-1, HER-2, HER-3, and HER-4. Some examples of glycoproteins on the surface of a cell include the folate receptors FR-α and FR-β.

Since receptors involved in the uptake of certain vitamins are overexpressed on cancer cells, vitamins are preferred examples of recognition modules for cancer treatment. Preferred examples of vitamins include biotin and folic acid.

A recognition module can also be, for example, a receptor-specific ligand. A receptor-specific ligand is a natural or synthetic molecule, such as a hormone (e.g., gastrointestinal peptidic hormones) or neurotransmitter, which specifically binds to a receptor on the surface of a cell. Some examples of receptor-specific ligands include bombesin and transferrin.

Preferably, the recognition module is an antibody or a functional equivalent of an antibody, such as a fragment of an antibody. More preferably, the antibody is a monoclonal antibody or a functional equivalent derived from a monoclonal antibody.

Suitable fragments of antibodies include any fragment that comprises a sufficient portion of the hypervariable region to bind specifically, and with sufficient affinity, to a molecule on the surface of a cell. Such fragments may, for example, contain one or both Fab fragments, or the F(ab′)₂ fragment. Preferably, the antibody fragments contain all six complementarity determining regions of the whole antibody, although functional fragments containing fewer than all of such regions, such as three, four or five CDRs, may also be suitable.

The preferred fragments are single chain antibodies, or Fv fragments. Single chain antibodies are polypeptides that comprise at least the variable region of the heavy chain of the antibody linked to the variable region of the light chain, with or without an interconnecting linker. These chains can be produced in bacteria or in eukaryotic cells.

The antibodies and functional equivalents can be members of any class of immunoglobulins, such as: IgG, IgM, IgA, IgD, or IgE, and the subclasses thereof. The preferred antibodies are members of the IgG1 subclass. The functional equivalents can also be equivalents of combinations of any of the above classes and subclasses.

Suitable variable and hypervariable regions of antibodies can be derived from antibodies produced by any mammal in which monoclonal antibodies are made. Some examples of suitable mammals include rabbits, rats, mice, horses, goats, and primates. Preferably, the monoclonal antibodies are derived from mice. The monoclonal antibodies thus obtained are humanized by methods known in the art for the purpose of human clinical use.

### Pharmaceuticals

Some examples of suitable pharmaceuticals include antitumor drugs, antiangiogenic drugs, multi-drug reversal agents, anti-inflammatory drugs, antibiotics antibacterial agents, antiparasitic drugs, and analgesics.

Examples of preferred antitumor drugs include taxoids. Examples of suitable taxoids include paclitaxel and docetaxel. Examples of preferred taxoids include the second-generation taxoids and advanced second-generation taxoids bearing modification at different positions including C-2, C-10 and C-3′ on the taxoid structure. These taxoids exhibit excellent activities particularly against multidrug resistant (MDR) cancer cell lines as well as tumors.

Additional examples of suitable taxoids include SB-T-1213 ((3′-dephenyl-3′-(2-methyl-1-propenyl)-10-propanoyldocetaxel)) and its congeners, SB-T-1102, SB-T-1103, SB-T-1104, SB-T-1212, SB-T-1214, SB-T-1216, SB-T-1217, SB-T-11033, SB-T-121303; SB-T-1250 ((3′-dephenyl-3′-(2,2-dimethyl-1-cyclopropyl)-10-acetyldocetaxel)); difluorovinyl-taxoids such as SB-T-12851, SB-T-12852, SB-T-12853 and SB-T-12854; SB-T-101131 (i.e., “Orataxel”); Also, see Ojima, I., et al., “Synthese and Structure-Activity Relationships of the Second-Generation Antitumor Taxoids: Exceptional Activity Against Drug-Resistant Cancer Cells,” J. Med. Chem. 39:3889-3896 (1996) and Ojima, I. et al., “Design, Synthesis and Biological Evaluation of New Generation Taxoids”, J. Med. Chem. 51, 3203-3221 (2008).

Further examples of taxoids include the compounds disclosed in U.S. Pat. No. 6,096,909; and U.S Patent Application Nos. 2002/0022651, 2003/0232878 and 2002/0087013. The aforementioned patent and applications are incorporated herein by reference in their entireties.

In some embodiments, polyunsaturated fatty acids (PUFAs) are linked to the C2′-position of the second generation taxoids. Examples of PUFAs include docosahexanoic acid (DHA), linolenic acid (LNA) and linoleic acid (LA). Preferred examples of such taxoids include DHA-SB-T-1214 and DHA-SB-T-1213.

Further, in some embodiments, albumin is covalently or non-covalently linked to an active compound to increase its efficacy.

Examples of other anti-cancer pharmaceuticals include platinum (IV) complexes, doxorubicin, daunorubicin, camptothecin, toptecan, monomethyllauristatin, mitomycins, maytansine, and maytansinoids.

The conjugates can comprises more than one type of pharmaceutical. For example, a multiple of different types of anticancer drugs can be placed on a single nanotube to effect combination therapies, i.e., different types of cancer drugs having different mechanisms of action and/or different intercellular targets can be placed on a single nanotube. For example, a conjugate can comprise a nanotube with both a taxoid and doxorubicin attached. Alternatively, various nanotubes each with different attached pharmaceuticals can be administered within a certain period of time to effect combination therapies.

### Linker Moieties

The pharmaceutical of the present invention is attached to the carbon nanotube by a linker moiety. The linker moiety is stable in blood circulation but readily cleavable in targeted cells, such as, for example, tumor cells.

Before a pharmaceutical is attached to a linker, it has at least one nucleophilic group capable of reacting with a carboxyl or thiocarboxyl group, or activated ester thereof, of the linker. Upon delivery of a conjugate to a target cell and internalization into the target cell via receptor-mediated endocytosis, the linker is efficiently cleaved by endogenous thiols. The free thiol group then undergoes intramolecular nucleophilic attack to form thiolactone, releasing the pharmaceutical in its active form.

Examples of suitable linker moieties include self-immolative disulfide-containing linkers. An example of a self-immolative disulfide-containing linker is a disulfide group attached to a functionalized alkyl group at one end and a phenylacetate group at the other end.

In a preferred embodiment, disulfide linkers are attached to the C-2′ position of taxoid molecules.

Amide linkers can also be used, for example, including alkyl-amide and polyoxyethylene-amide.

### Methods of Making the Conjugates

In another aspect, the present invention provides methods of making the conjugates described above.

In the methods, a plurality of carbon nanotubes with oxygen moieties can be produced or obtained from an outside source. The density of oxygen moieties on a nanotube, and the positions of the moieties on a nanotube, can be controlled by the manner selected to oxidize the nanotubes. Examples of different methods of obtaining nanotubes with oxygen moieties follow.

In one embodiment, a minimum amount of oxygen moieties are placed on nanotubes. In this embodiment, the nanotubes are not oxidized. Some oxygen moieties arise on carbon nanotubes during their formation.

In another embodiment, carbon nanotubes with oxygen moieties are produced by oxidation processes. Processes for oxidizing nanotubes are well known in the art. For example, carboxyl groups can be introduced by a reactions with peroxides of dicarboxylic acid (Peng et al., J. Am. Chem. Soc. 125:15174 (2003)), potassium permanganate (Banerjee et al., Nano Letters 2:49 (2002)), or ozone (Banerjee et al., J. Phys. Chem. B 106:12144 (2002)).

For example, a moderate level of oxygen moieties can be placed on the nanotubes. e.g., about 5% to about 10% of the carbon atoms on a nanotube have attached oxygen moieties. Examples of methods of oxidation wherein oxygen moieties are preferentially placed on the end caps and/or defect sites of nanotubes follow. Raw SWNT bundles can be oxidized according to existing procedures involving acidic potassium permanganate solution and hydrochloric acid. See for example Hiura et al. Adv. Mater 7:275 (1995). SWNT samples can be prepared via arc discharge, pulsed laser vaporization, or chemical vapor deposition. The SWNT samples can be purified by sonication and filtration through 0.8 micron pore membranes. See for example, Bonard et al. Adv. Mat., 9:827 (1997); Tohji et al. J. Phys. Chem. B, 101:1974 (1997); and Tohji et al., Nature, 383:679 (1996). Other examples of moderate oxidation methods include wet air-oxidization; gaseous phase oxidation; acid phase oxidation, e.g., nitric acid oxidation; and other types of liquid phase oxidation.

U.S. Pat. No. 7,189,455 includes methods of controlling the type of oxygen moieties placed on carbon nanotubes. The patent is incorporated herein by reference in its entirety. The methods comprise contacting ozonized carbon nanotubes with a particular type of cleaving agent. For providing carbon nanotubes comprising predominately carboxylic and/or esteric moieties, peroxides are used as the cleaving agent. This oxidizing method allows for different densities of oxygen moieties to be placed on the nanotubes. The density can range from about 1 to about 35% of the carbon atoms on the nanotubes having an oxygen moiety. A high level of oxygen moieties is about 25 to 35% of the carbon atoms on the nanotubes have an oxygen moiety.

Optionally, the carbon nanotubes can be shortened. Techniques by which to shorten nanotubes include acid etching, ion beam milling, ball milling, and gas etching, as would be known by a skilled artisan.

The pharmaceuticals and recognition modules are then placed anywhere on the plurality of carbon nanotubes.

In a preferred embodiment, the pharmaceuticals are substantially selectively attached to the sidewalls; and the recognition modules are substantially selectively attached to the ends/defect sites of the carbon nanotubes. In this embodiment, two different bifunctional amines having different protecting groups are provided. A first protecting group is attached to a first bifunctional amine; and a second protecting group is attached to a second bifunctional amine. The nanotubes are then reacted with each type of bifunctional amine consecutively.

For example, in one embodiment, first, the plurality of carbon nanotubes are contacted with a first bifunctional amine to yield a plurality of carbon nanotubes with bifunctional amine groups primarily attached to sidewalls. The attachment of the first bifunctional amine to the sidewalls can be accomplished by methods known in the art. For example, attachment of a first bifunctional amine can be accomplished by 1,3-dipolar cycloaddition of azomethine ylide generated by condensation of an amino acid and an aldehyde (Georgakilas et al., JACS 124(5):760-761 (2002)). Alternatively, attachment of a bifunctional amine can be accomplished by [2+1]cycloaddition of nitrenes.

Second, the sidewall-functionalized nanotubes are reacted with a second bifunctional amine to substantially place second bifunctional amine groups at the end/defect sites of the nanotubes. The attachment of the second bifunctional amine can be accomplished by methods known in the art. For example, the nanotubes can be reacted with N-(4-aminobutyl)phthalimide to obtain a phthalimide-protected amine on the end/defect sites. Alternatively, the nanotubes can be reacted with a 9-fluorenylmethyloxycarbonyl (FMOC) group to obtain a fluorenylmethyloxycarbonyl-protected amine on the end/defect sites.

In another embodiment, a bifunctional amine is first substantially placed on the sidewalls and a different bifunctional amine is next substantially placed on the end/defect sites.

Next, the plurality of nanotubes, having bifunctional amines with different protecting groups, is consecutively reacted with recognition moieties and pharmaceuticals. The recognition moieties and pharmaceuticals each replace different protecting groups. The order of the reactions is not critical. Conjugates with covalently attached recognition moieties and covalently attached pharmaceuticals are yielded. The pharmaceuticals are attached via linkers. Preferably, the recognition moieties are primarily attached to the end/defect sites and the pharmaceuticals are primarily attached to the sidewall.

Following are some examples of protecting groups that can be used in the present method: carbobenzyloxy (Cbz) group (removed by hydrogenolysis); tert-butyloxy-carbonyl (BOC) group (common in solid phase peptide synthesis; removed by concentrated, strong acid (e.g., HCl or CF₃COOH)); 9-fluorenylmethyloxycarbonyl group (common in solid phase peptide synthesis, removed by base, such as piperidine); benzyl (removed by hydrogenolysis); p-methoxyphenyl group (removed by ammonium cerium (IV) nitrate).

For example, in one embodiment, the nanotubes are reacted with a recognition module at slightly basic conditions so that the phthalimide or FMOC groups are replaced with recognition modules.

### Activation of the Pharmaceuticals

Many pharmaceuticals are cytotoxic. For example, systemic toxicity associated with conventional chemotherapy is well known. One of the many benefits of the present invention is that pharmaceuticals which are cytotoxic are rendered substantially non-cytotoxic when part of the conjugates. For example, there is a substantial loss of potency of taxoids when made part of the conjugate, rendering the taxoid systemically non-toxic in the circulatory system. Once the conjugates reach their target cells, the pharmaceuticals are internalized in the cells and activated. For example, upon internalization into cancer cells, the conjugate is readily cleaved in situ to release the active cytotoxic pharmaceutical which leads to its pharmaceutical effect, e.g., tumor cell death.

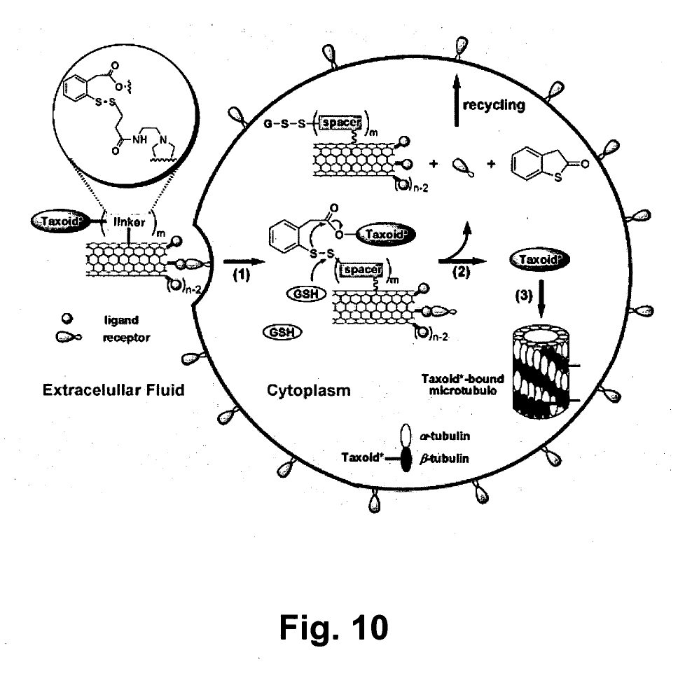

While not wanting to be bound to a mechanism of action, FIG. 10 illustrates the probable three key steps involved in the activation of the conjugate. In FIG. 10, biotin is the recognition module, and a taxoid is the pharmaceutical. First, the biotin-nanotube-linker-taxoid conjugate is internalized into the tumor cells through receptor-mediated endocytosis. The biotin moieties covalently attached to the nanotube efficiently recognizes the biotin receptors overexpressed on the tumor cell surfaces. The presence of multiple biotin moieties, localized at the ends of the nanotube, enhances the internalization of the conjugate via increased probability for receptor binding or via multivalent binding (Hong et al., Chem. Biol. 14: 107-115 (2007)).

Second, the active form of the pharmaceutical is released through cleavage of the disulfide bond in the linker moiety by endogenous thiols. An example of an endogenous thiol is glutathione (GSH). Cleavage of the disulfide bond generates a sulfhydryl group which subsequently undergoes a thiolactonization process to form benzothiophen-2-one and regenerates a free taxoid in its active form.

Concentrations of GSH are typically 1-2 μM in circulating human blood plasma, but are in the range of 2-8 mM in tumor tissue (Meister, A. (1983) Metabolism and transport of glutathione and other g-glutamyl compounds (Raven Press, New York). Zheng et al., Bioconjugate Chem. 16: 598-607 (2005)). Thus, the adventitious activation Of a cytotoxic pharmaceutical is minute in the blood circulation, whereas the activation process would be facile in the tumor cells.

Third, the as-released taxoid binds to microtubules, inhibiting cell mitosis at the G2/M stage by stabilizing microtubules, which triggers signaling to cause apoptosis (Jordan et al. Chem. Biol. 9: 93-101 (2002); Nogales et al., Nature (London, United Kingdom) 375: 424-427 (1995)).

The conjugates of the present invention serve as drug delivery platforms which offer (a) biomarker-targeted drug delivery, (b) delivery of greater therapeutic payloads as single molecular entity and (c) use of multiple drug warheads for combination therapy.

For the pharmaceutical purposes described above, the conjugates of the invention can be formulated in pharmaceutical preparations optionally including a suitable pharmaceutical carrier (vehicle) or excipient. In this specification, a pharmaceutical carrier is considered synonymous with a vehicle or an excipient as understood by practitioners in the art.

Preferably, the conjugate pharmaceutical formulations are administered systemically. Systemic administration includes enteral or parenteral modes of administration, e.g., intravenous; intramuscular; subcutaneous; or intraperitoneal. For example, the conjugate formulations may be administered by injection of a solution or suspension; or intrabronchially in the form of, for example, an inhaler spray.

The conjugate formulations may comprise one or more of the following: a stabilizer, a surfactant, a salt, a buffering agent, or a combination thereof. The stabilizer may be, for example, an amino acid, such as glycine; or an oligosaccharide, such as sucrose, tetralose, lactose or a dextran. Alternatively, the stabilizer may be a sugar alcohol, such as mannitol; or a combination thereof.

Some examples of suitable surfactants include Tween 20, Tween 80; Cremophor, Solutol H-15; a polyethylene glycol or a polyoxyethylene polyoxypropylene glycol, such as Pluronic F-68 at from about 0.001% (w/v) to about 10% (w/v). The salt or buffering agent may be any salt or buffering agent, such as, for example, sodium chloride, or sodium/potassium phosphate, respectively.

For intramuscular, intraperitoneal, subcutaneous and intravenous use, sterile solutions of the conjugates can be employed, and the pH of the solutions can be suitably adjusted and buffered. For intravenous use, the total concentration of the solute(s) can be controlled in order to render the preparation isotonic.

The conjugate formulations may additionally contain one or more conventional additives. Some examples of such additives include a solubilizer such as, for example, glycerol; an antioxidant such as, for example, benzalkonium chloride (a mixture of quaternary ammonium compounds, known as “quart”), benzyl alcohol, chloretone or chlorobutanol; an anaesthetic agent such as, for example, a morphine derivative; an isotonic agent, or a combination of these. For aqueous suspensions, emulsifying agents, suspending agents, or a combination thereof, may be added. As a precaution against oxidation or other spoilage, the conjugate formulation may be stored under nitrogen gas in vials sealed with impermeable stoppers.

The conjugates may be administered alone or as an adjunct with other conventional drugs for treating conditions or diseases, including cancer.

The timing of the administration of the conjugate formulations is any timing that would provide effective treatment, as would be known by a skilled artisan. For example, the formulations may be administered intermittently or by controlled release. Controlled release administration is a method of drug delivery to achieve a certain level of the drug over a particular period of time.

The present invention includes methods of treatment of diseases or disorders by administration of an effective amount of the conjugates described throughout this specification.

### EXAMPLES

### Experimental Section

### 1. Syntheses of Functionalized SWNTs

Chemicals and reagents, if not specified, were used as received from the relevant commercial sources. Compound 5 and 7 were prepared according to prior literature. (Kordatos et al., J. Org. Chem. 2001, 66, 4915-4920; Varghese et al., J. Med. Chem. 2005, 48, 6350-6365.) Solvents were purified either by distillation after drying with respective dessication agents according to either standard protocols or by using PureSolv™ (Innovative Technology, Inc) under N₂. De-gassed solvents were used when necessary. TLC was performed on Merck DC-aluminfolien using a Kieselgel 60F-254 instrument. ¹H NMR spectra were measured on either a Varian 300 or Varian 400 NMR spectrometer. Infrared spectra were obtained on a Nexus 670 (Thermo Nicolet) equipped with a single reflectance ZnSe ATR accessory, a KBr beam splitter, and a DTGS KBr detector. UV-vis spectra were recorded on a UV1 (Thermo Spectronic) spectrometer. For transmission electron microscopy (TEM), samples were prepared by drying droplets of sample dispersion onto 300 mesh carbon coated copper grids (Ted Pella). Low magnification TEM images were taken at an accelerating voltage of 80 kV on a FEI Tecnai 12 BioTwinG² instrument, equipped with an AMT XR-60 CCD Digital Camera System.

Preparation of oxidized SWNT (4). Pristine SWNTs (Carbon Nanotechnologies Inc.) used in this experiment were CNI grade (Lot No. P0279). Per product specifications, mean diameters of the SWNTs were about 1 nm and corresponding lengths ranged between 300 and 1000 nm. Accurate SWNT length and diameter determination after functionalization was hampered because of the presence of aggregation of the dispersed tubes. Pristine SWNTs (10 mg) were oxidized to yield a functionalized SWNT 4 using 5 mL of a 3:1 (v/v) concentrated H₂SO₄ and HNO₃ solution by sonicating at 40° C. for 2 h. followed by heating at 70° C. for 30 min. The reaction mixture was diluted to 200 mL with water and filtered through a 0.2 μm polycarbonate membrane. The product was then washed extensively by water until the pH reached neutral conditions and further oven dried at 120° C. under vacuum for 2 h (9 mg, 90% yield).

Synthesis of SWNT-FITC (1). A suspension of oxidized SWNT 4 (15 mg) in DMF (5 mL) was added N-(2-N-Boc-ethyl)glycine (5) (70 mg, 0.32 mmol) and paraformaldehyde (47 mg, 1.57 mmol). (Kordatos et al., J. Org. Chem. 2001, 66, 4915-4920.) The reaction mixture was subsequently heated overnight at 125° C. under a N₂ atmosphere. Excess amino acid 5 and paraformaldehyde were removed by filtration. The resulting residue was further purified by precipitation with methanol/ether 5 times (4/1, 12 mL/3 mL) and dried under vacuum to afford product 3 (11 mg, 73% yield). The resulting functionalized SWNT 6 was treated with 2 mL of TFA/CH₂Cl₂ (1:1) and then stirred at room temperature for 2 h. The solvent was evaporated in vacuo. Crude product was purified by washing with several aliquots of methanol and diethyl ether 5 times (4/1, 12 mL/31 mL), and subsequent drying under vacuum. The amount of loading of functional groups per gram was estimated via a quantitative Kaiser test. To a solution of functionalized SWNTs obtained above (10 mg, 1.8 μmol, based on the loading calculated using the quantitative Kaiser test) in 1 mL of DMF, a solution of FITC (50 mg, 0.13 mmol) and DIPEA (0.1 mL) in 1 mL of DMF was added. The mixture was then stirred overnight at room temperature. Upon solvent removal, the resulting SWNT-FITC (1) was reprecipitated 5 times from methanol/ether (4/1, 12 mL/3 mL) and finally dried under vacuum for 5 h to give the pure conjugate 1 (7 mg, 70% yield).

Synthesis of biotin-SWNT-FITC (2). A solution of functionalized SWNT 4 (20 mg), N-(4-aminobutyl)phthalimide (7) (100 mg, 0.46 mmol), DIPEA (0.3 mL), and HATU (175 mg, 0.46 mmol) in 2 mL of anhydrous DMF was stirred at room temperature for 3 h. (Varghese et al., J. Med. Chem. 2005, 48, 6350-6365.) An excess of amine 7 was removed by washing 5 times with methanol/ether (4/1, 12 mL/3 mL) to afford modified SWNT 8. A mixture of 10 mg of modified SWNT 8 and of hydrazine hydrate (25%, 0.2 mL) in 5 ml of ethanol was heated overnight under reflux in a nitrogen atmosphere. The resulting phthalhydrazide was removed by dialysis to yield amine-functionalized SWNT 9. The loading of amine groups per gram was estimated using the quantitative Kaiser test.

Amine-functionalized SWNT 9 (10 mg, 5 μmol, based on the loading calculated with the quantitative Kaiser test) and Biotin-OSu (10, 110 mg, 0.32 mmol) were suspended in 3 mL of anhydrous DMSO. The resulting suspension was stirred overnight at room temperature. Excess Biotin-OSu was removed by dialysis to yield biotin-N-Boc-SWNT conjugate 11 (10 mg, quant.).

SWNT conjugate 11 was treated with 2 ml of TFA/CH₂Cl₂ (1:1) and the reaction mixture was stirred at room temperature for 2 h. Upon evaporation of the solvent in vacuo, the crude product was washed with methanol/ether 5 times (4/1, 12 mL/3 mL) and dried under vacuum. To a suspension of the resulting biotin-amine-SWNT conjugate 12 (10 mg, 1.8 μmol, based on the loading calculated with the quantitative Kaiser test) in 2 ml of anhydrous DMF was added FITC (50 mg, 0.13 mmol) and DIPEA (0.1 mL). The resulting mixture was stirred overnight at room temperature. The excess of FITC was removed by washing 5 times with methanol/ether (4/1, 12 mL/3 mL), and the resulting product was dried at room temperature under vacuum for a few hours to afford biotin-SWNT-FITC conjugate 2 (7.5 mg, 75% yield).

Synthesis of biotin-SWNT-linker-taxoid(SB-T-1214)-fluorescein (3). A suspension of biotin-amine-SWNT 12 (10 mg, 1.8 μmol, based on the loading calculated with the quantitative Kaiser test) in 4 mL of anhydrous DMF was added SuO-linker-taxoid-fluorescein 13 (160 mg, 0.099 mmol) and DIPEA (0.1 mL). The resulting mixture was stirred overnight at room temperature. Excess 13 was removed by washing 5 times with methanol/ether (4/1, 12 mL/3 mL) and the product was ultimately dried at room temperature under vacuum for 5 h to afford biotin-SWNT-linker-taxoid-fluorescein conjugate 3 (6 mg, 60% yield).

### 2. Biological Experiments

Cell culture. L1210 (ATCC) and L1210FR (a gift from Dr. Gregory Russell-Jones, Access Pharmaceuticals Australia Pty Ltd., Targeted Delivery, Unit 5, 15-17 Gibbes St, Chatswood, NSW, Sydney 2067. Australia) were grown as suspension in a RPMI-1640 cell culture medium (Gibco) in the absence of folic acid (FA) supplemented with 10% (v/v) heat-inactivated fetal bovine serum (FBS) and 1% (v/v) Penicillin and Streptomycin (P/S) at 37° C. in humidified atmosphere with 5% CO₂. W1-38 embryonic fibroblast cells (ATCC) were cultured as monolayers on a 100 mm tissue culture dishes in a DMEM cell culture medium (Gibco) supplemented with 10% (v/v) heat-inactivated FBS and 1% (v/v) P/S at 37° C. in humidified atmosphere with 5% CO₂. The cells were harvested, collected by centrifugation at 1000 rpm for 6 min, and resuspended in fresh medium at different cell density for biological experiments.

Incubation of cells with SWNT conjugate 1 and 2. The cell suspension (1 mL) at 5×10⁵ cells/mL was initially added to a microtube. The SWNT conjugates 1 or 2 (10 μL) in DMSO were then injected to the microtube to provide for a final concentration of 10 μg/mL and the resultant suspension was incubated at 37° C. for 3 h. For low temperature experiments, the incubation of cells (L1210FR) with conjugates 1 or 2, was carried out in the cold room at 4° C. for 3 h. For the NaN₃ study, the cells were initially pre-incubated at 37° C. for 0.5 h with 0.05% (v/v) of NaN₃, before the addition of the SWNT conjugates 1 or 2. For the biotin study, prior to incubation with the conjugates, the cells were treated with 2 mM of biotin (at final concentration) at 37° C. for 0.5 h.

Incubation of cells with conjugate 3. The cell suspension (1 mL) at 5×10⁵ cells/mL was initially added to a microtube. The conjugate 3 (10 μL) in DMSO was subsequently added to the microtube at a final concentration of 50 μg/mL. After incubation at 37° C. for 3 h, the cells were washed twice by PBS to remove excess conjugates and resuspended in the medium. For observation of the release of taxoid in a short period of time, glutathione ethyl ester (10 μL) was then added to the L1210 FR cell suspension at a final concentration of 2 mM and incubated for another 2 h. The excess glutathione ethyl ester was removed by washing twice with PBS and the cells were then resuspended in 100 μL PBS prior to imaging. In the control experiment, DMSO (10 μL) was then added to the suspension and incubated for another 2 h. After incubation, the cells were washed with PBS, collected by centrifugation twice, and resuspended in 100 μL PBS prior to imaging.

Immunofluorescence. The cell suspension (1 mL) at 5×10⁵ cells/mL was initially added to a microtube. The conjugate 3 (10 μL) in DMSO was subsequently added to the microtube at a final concentration of 50 μg/mL and incubated at 37° C. for 12 h. Cells were extracted in PEM microtubule stabilizing buffer [100 mM PIPES, 2 mM EGTA, and 2 mM MgCl₂ (pH 6.8)] for 4 min, fixed in 3% formaldehyde in PEM for 40 min, blocked with 20% normal goat serum for 30 min and incubated with 1:100 α-tubulin monoclonal antibody (Aldrich) for 1 h. After removal of the excess primary antibody with PBS, the Texas Red® goat anti-mouse IgG (Invitrogen, 1:100) was used as the secondary antibody and incubated for 1 h. The excess IgG was removed by washing the cells with PBS. The cells were resuspended in 100 μL PBS for imaging.

Confocal microscopy imaging. The treated cells mentioned above were resuspended in 100 μL of PBS after each experiment, and dropped onto an uncoated bottom glass dish (MatTek Corp.). CFM experiments were performed using a Zeiss LSM 510 META NLO two-photon laser scanning confocal microscope system, operating at the 488 nm excitation wavelength and detecting emission wavelengths using a 505-550 nm bandpass filter. Images were captured with a C-Apochromat 63×/1.2 Water (corr.) objective or a Plan-Apochromat 100×/1.45 oil objective. Acquired data were analyzed using LSM 510 META software.

Flow cytometry fluorescent measurements. The treated cells mentioned above were resuspended in 0.5 mL of PBS. Cells were analyzed using a flow cytometer. FACSCalibur, operating at the 488 nm excitation wavelength and detecting emission wavelengths using a 530/30 nm bandpass filter. At least 10,000 cells were counted for each experiment using CellQuest 3.3 software (Becton Dickinson) and the distribution of the FITC fluorescence was analyzed using the WinMDI 2.8 freeware (Joseph Trotter, Scripps Research Institute). The propidium iodide staining was used in all experiments to rule out the dead cells in the flow cytometry analysis.

Cytotoxicity assay of conjugate 3. The cells were harvested, collected, and resuspended in 100 μL at a concentration of 2×10⁴ cells per well in 96-well plates. For the adhesive cell type, the cells were allowed to reseed to the bottom of the plates overnight and the fresh medium were added to each well upon removal of the old medium. The SWNT conjugates were diluted to a series of concentration in medium with FBS as stock solution. The stock solution containing SWNT conjugates (10 μL) was added to each of the wells in the 96-well plates and the cells cultured for 3 days. At the end of this time, the number of viable cells each wells was determined by a quantitative colorimetric staining assay using a tetrazolium salt (MTT, Sigma Chemical Co.). The inhibitory concentration (IC₅₀) of each compound was determined, as the concentration required inhibiting 50% of the growth of the L1210FR cells.

### Results

### 1. Synthesis and Characterization

To demonstrate the specificity and efficacy of the SWNT-based conjugate, three fluorescently labeled SWNT-conjugates (1, 2, and 3) were designed and synthesized, as shown in FIG. 10. Conjugates 1 and 2 were labeled with fluorescein isothiocyanate (FITC). The resulting fluorescent conjugates, SWNT-FITC (1) and biotin-SWNT-FITC (2), were used to track the internalization of SWNT and biotin-SWNT, respectively, into the tumor cells. Biotin-SWNT-linker-taxoid-fluorescein (3) is the designed fluorescent molecular probe of the SWNT-based conjugate for the receptor-mediated endocytosis and intracellular drug release, illustrated in FIG. 10.

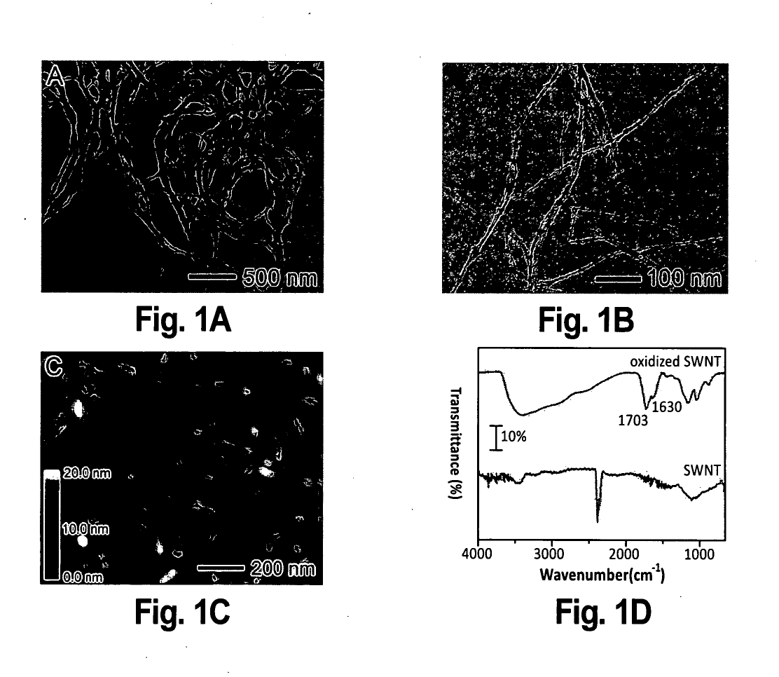

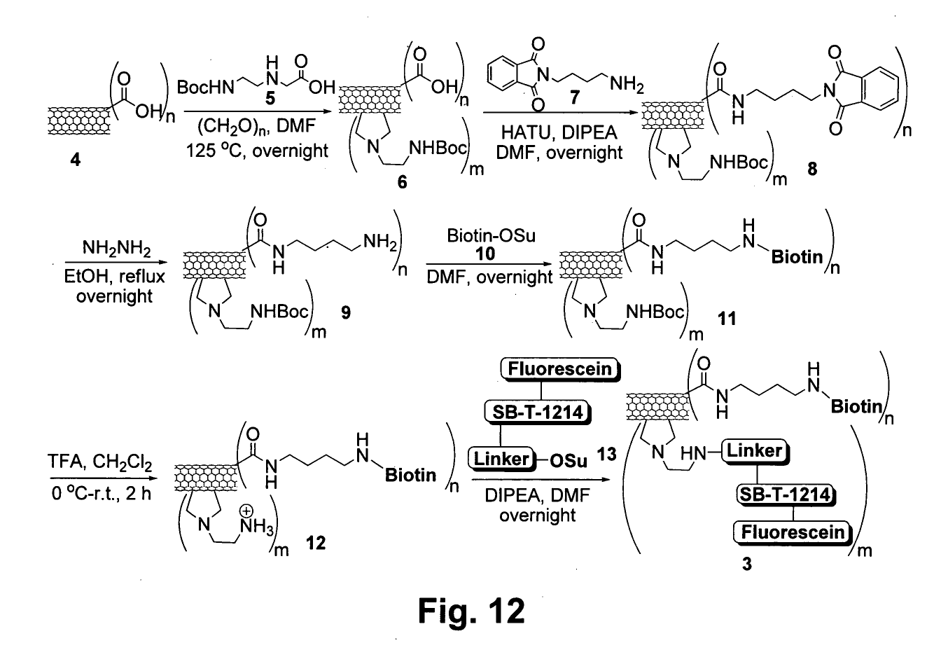

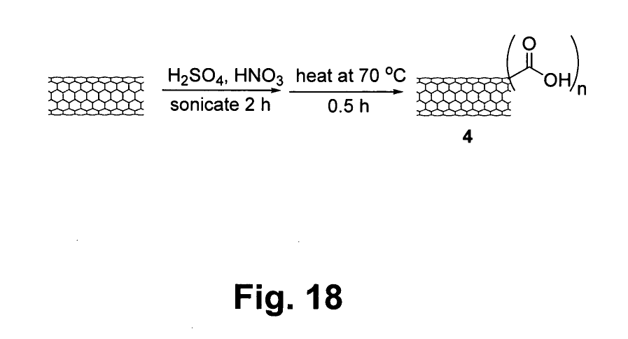

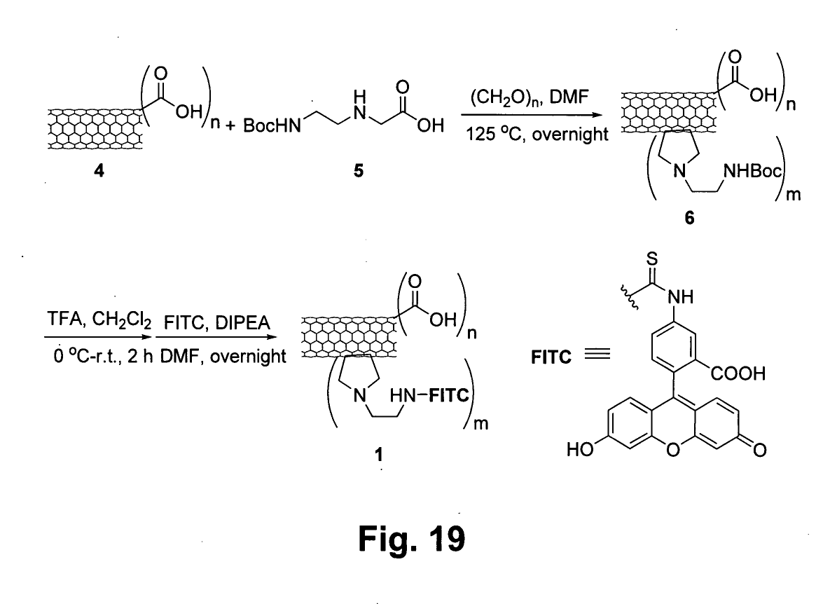

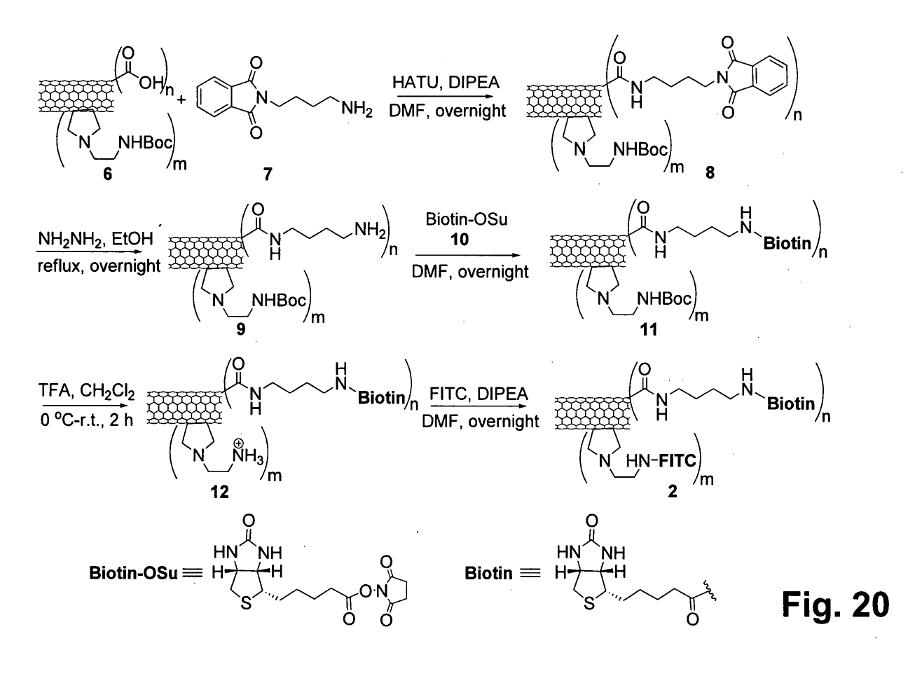

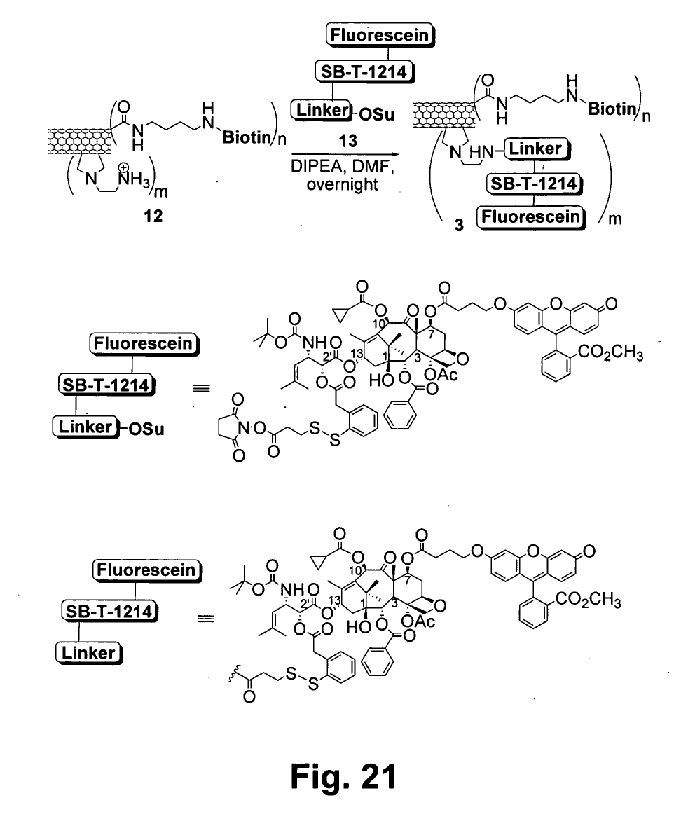

For the syntheses of conjugates 1, 2 and 3, it was detailed in the experimental section and FIGS. 18-21. Briefly, FIG. 12 illustrates the synthetic pathway to conjugate 3 from the oxidized SWNT (4). A batch of pristine HiPco SWNT (0) was first functionalized and purified by oxidation in concentrated H₂SO₄:HNO₃ (3:1 by volume) with sonication for 2 h, followed by reflux at 70° C. for 30 min. (Liu et al., Science 1998, 280, 1253-1256.) FIGS. 1A and 1B show the TEM image of the HiPco SWNTs before and after oxidation. The ends and defect sites on the side walls of the oxidized SWNTs were functionalized with carboxylic acid and carboxylate groups, whose presence was confirmed by ATR IR spectroscopy, showing relevant expected peaks at 1703 and 1630 cm⁻¹ (FIG. 1D). FIG. 1C show the AFM image of the oxidized SWNTs with ˜3 nm in diameter and ˜250 nm in length. These carboxylic groups were subsequently converted to amide groups through condensation with amines. Tube sidewalls were functionalized with amine moieties through 1,3-dipolar cycloaddition of azomethine ylide in situ generated. (Pastorin et al., Chem. Comm. 2006, 1182-1184.) The extent of amine loading was estimated to be 0.5±0.03 and 0.2±0.02 mmol per gram at the ends/defect sites and the sidewalls of SWNTs, respectively, by means of the Kaiser test. (Sarin et al., Anal. Biochem. 1981, 117, 147-157.) Finally, biotin molecules and the fluorescein-labeled taxoid-linker moieties were conjugated to the amine moieties located at the ends/defect sites and sidewalls of SWNTs, respectively, through the standard peptide coupling reactions to yield the desired conjugate, biotin-SWNT-linker-taxoid-fluorescein (3). In principle, these couplings using modifiers in large excess should proceed quantitatively to give a maximum of 714 biotin modules (at the ends and the defect sites on the side walls) and 285 taxoid modules (on the side walls themselves) per SWNT (based on the mass of a carbon nanotube with 1 μm in length and 1 nm in diameter estimated as 2.2×10⁻¹⁸ g). (Zhu et al., J. Am. Chem. Soc. 2005, 127, 9875-9880.) Therefore, conjugate 3 of 100 μg/mL is estimated to contain taxoid molecules of 13.9 μM.

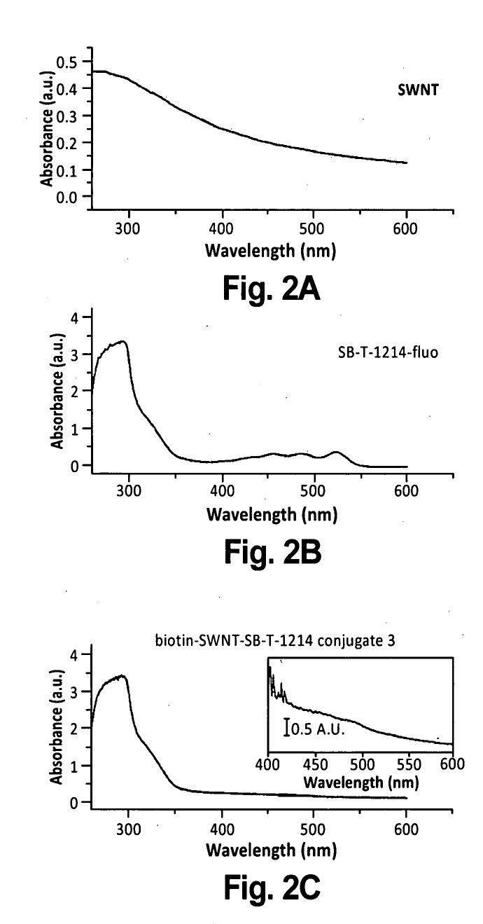

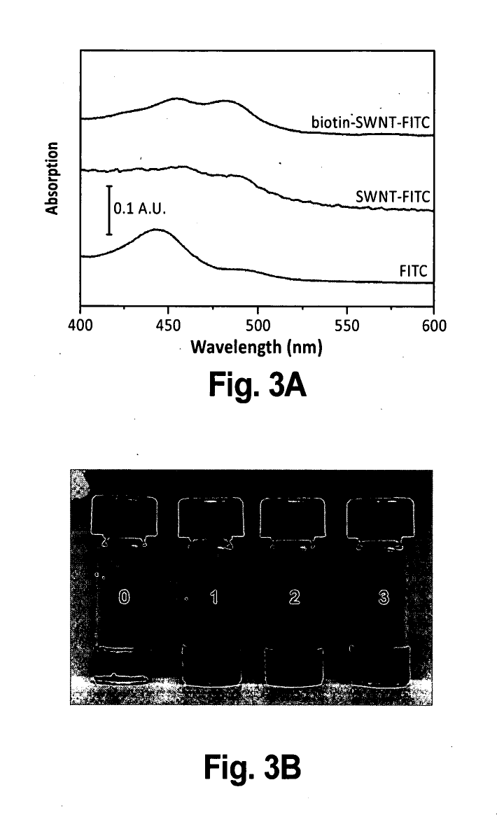

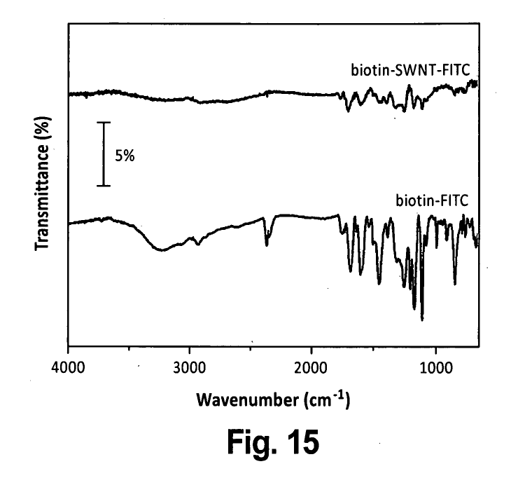

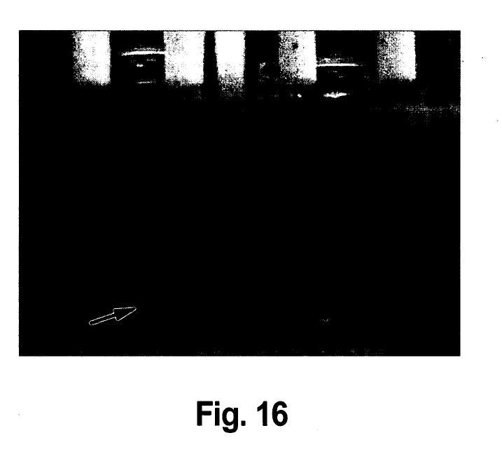

Conjugate 3 was analyzed by UV-visible spectroscopy (FIG. 2). The peak at ˜280 nm can be attributed from sum of absorption arising from taxoid (SB-T-1214) molecules and the dye molecules (fluorescein), while absorption peaks at 455, 485 and 524 nm are characteristic of fluorescein. Conjugate 1 was prepared from functionalized SWNT 6 by attaching the FITC groups to its sidewall through deprotection of the Boc group and the addition of FITC. Conjugate 2 was prepared from conjugate 1 by introducing the biotin moiety in the same manner as that used for the synthesis of conjugate 3. Conjugates 1 and 2 were characterized by UV-visible spectroscopy (FIG. 3A). Absorption peaks at ˜450 and ˜490 nm confirmed the presence of FITC in these conjugates. The discrepancy of the peak position and intensity between samples may be caused by the pH of the suspension. (Sjoback et al., Sepctochimica Act. Part A 1995, 51, L7-L21.) The presence of FITC and biotin was also confirmed by the ATR IR spectroscopy of conjugate 2 (FIG. 15). It is worthy of note that the solubility of the functionalized SWNTs (1, 2, and 3) in polar solvent, i.e. dichloromethane, was greatly enhanced as compared with pristine tubes (0), as shown in FIG. 3B. The conjugate 3 are also dispersed well in cell culture medium at 50 μg/mL for months (FIG. 16).

### 2. Internalization of SWNT Conjugate 1 and Biotin-SWNT Conjugate 2

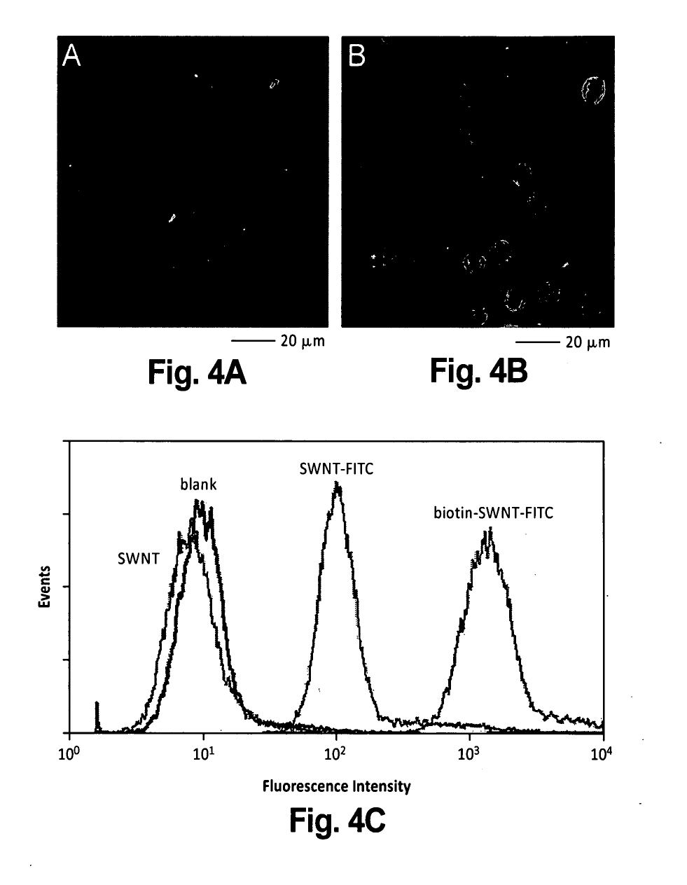

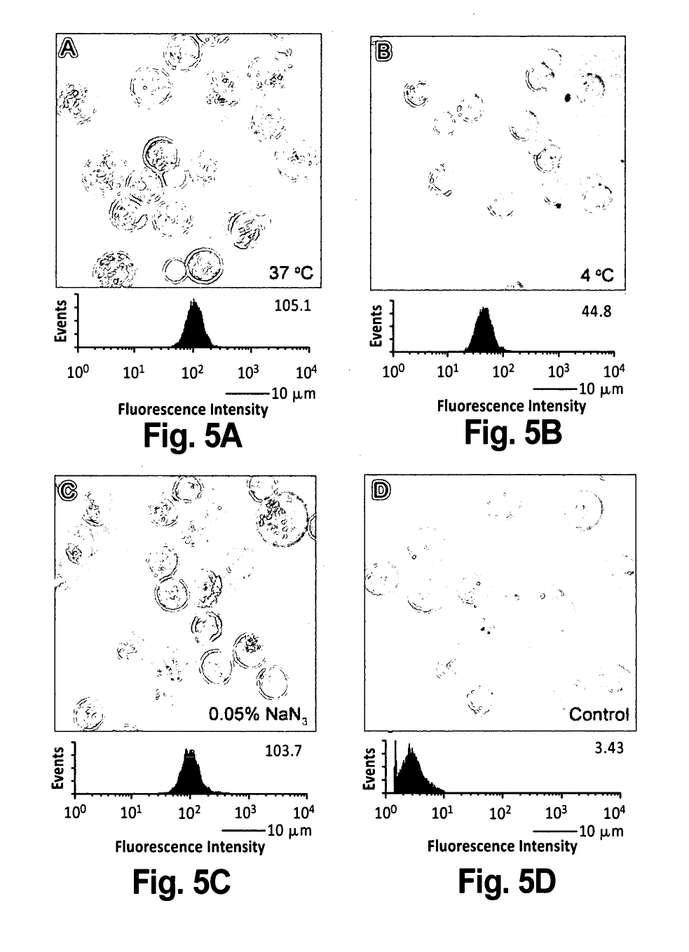

Cellular uptake of conjugates 1 and 2 was examined by a leukemia cell line, L1210FR, which overexpresses biotin receptors on its surface. (Russel-Jones et al., J. Inorg. Biochem. 2004, 98, 1625-1633.) FIGS. 5A and 5B show confocal fluorescence microscopy (CFM) images of L1210FR cells after treatment with 10 μg/ml, (final concentration) of SWNT-FITC (1) and biotin-SWNT-FITC (2) conjugates, respectively, for 3 h at 37° C. The treated leukemia cells were washed with phosphate buffered saline (PBS) to remove excess fluorescent probes in extracellular medium. L1210FR cells treated with conjugate 2 yielded far more intense fluorescence than those incubated with conjugate 1. This observation can be attributed to the remarkably increased permeability of conjugate 2 into the cancer cells because of the highly effective interaction of biotin and its receptors on the leukemia cells. Flow cytometry data (FIG. 4C) on average of 10,000 treated live cells also supported this observation, i.e., the fluorescence intensity of the biotin-SWNT conjugate 2 increased one order of magnitude compared to the SWNT conjugate 1.

The mechanism of internalization of SWNTs into cells has not been fully established. It has been proposed that SWNTs wrapped with proteins or genes can be internalized into cells via endocytosis, whereas SWNTs functionalized with small molecules tend to act as nanoneedles that can pierce cell membranes, thereby allowing for their diffusion into cells. (Kam et al., Angew. Chem. Int. Ed. 2006, 45, 577-581; Kostarelos et al., Nat. Nanotech. 2007, 2, 108-113.) Endocytosis is known to be energy dependent and could be hindered at low temperature and in the presence of the metabolism inhibitor, such as NaN₃. (Mukherjee et al., Physiol. Rev. 1997, 77, 759-803; Silverstein et al., Annu. Rev. Biochem. 1977, 46, 669-722; Schmid et al., J. Cell Biol. 1990, 111, 2307-2318.) To probe the mechanism of cellular uptake in the SWNT conjugates, the L1210FR cells were incubated with conjugates 1 at 4° C. or in the presence of 0.05% NaN₃ for comparison with the uptake at 37° C. It was found that the conjugates 1 were able to transverse the cell membrane at low temperature or in the presence of 0.05% NaN₃ for 3 h incubation. Similar fluorescent intensity were visualized after incubation without (FIG. 5A) or with (FIG. 5C) 0.05% NaN₃ presence. A slightly decreased, i.e. 2 times, in the fluorescent intensity was observed in low temperature (FIG. 5B) due to the temperature effect of diffusion process. In control experiment, the cells with treatment of 10 μL DMSO shows no fluorescence at all (FIG. 5D). The flow cytometry analysis on 10,000 cells is also in agreed with these findings. The internalization of SWNT itself to the cells is temperature-related, but energy-independent.

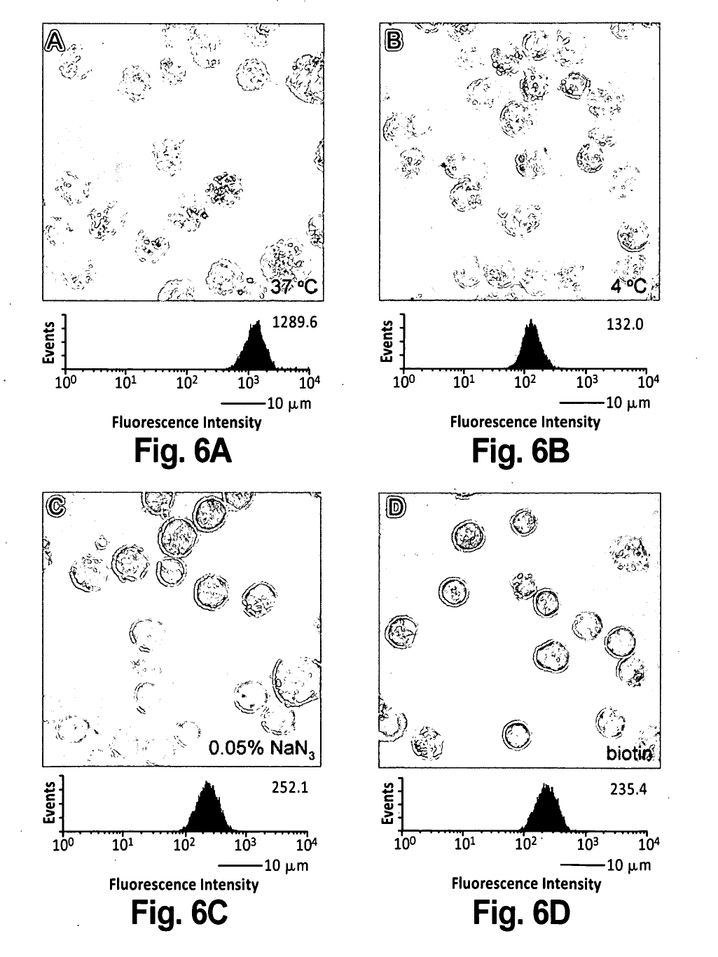

The biotin uptake is known to be temperature and energy dependent receptor mediated endocytosis. (Balamurugan et al., Am. J. Physiol. Renal. Physiol. 2005, 288, F823-F831; Becker et al., Proc. Nat. Acad. Sci. 1971, 68, 2604-2607.) To examine the mechanism of the cellular uptake of the biotin-SWNT conjugates, conjugates 2 were incubated at different condition with L1210FR cells that are over-expressed with biotin receptor on their surface. The fluorescent intensity of cells incubated at 4° C. decreased by one order of magnitude as compared with that of the ones treated at 37° C., as shown in FIGS. 6A and 6B. It is implied that the internalization of the conjugate 2 was hindered at low temperature. This observation was not only caused by the temperature effect on the SWNT internalization, but also on the endocytosis of the biotin functionalities. FIG. 6C shows that the fluorescent intensity decreased dramatically in the present of 0.05% NaN₃, indicating that endocytosis of biotin conjugates is energy-dependent and can be blocked by the NaN₃. To further verify the nature of the internalization of biotin-conjugate 2 as that of receptor-mediated endocytosis, L1210FR cells were incubated with excess biotin to saturate accessible biotin receptors on the surfaces of the leukemia cells, and then treated them with biotin-conjugate 2 at 37° C. for 3 h. The CFM image in FIG. 6D clearly indicates a drastic reduction in the fluorescence intensity, as compared to that observed in the absence of excess biotin (FIG. 6A). These results confirm that the receptor-mediated endocytosis is by far the predominant mechanism of internalization, with nanotube diffusion as a contributing, albeit relatively minor pathway to the observed data.

3. Release of Taxoid from Conjugate 3 In Vitro

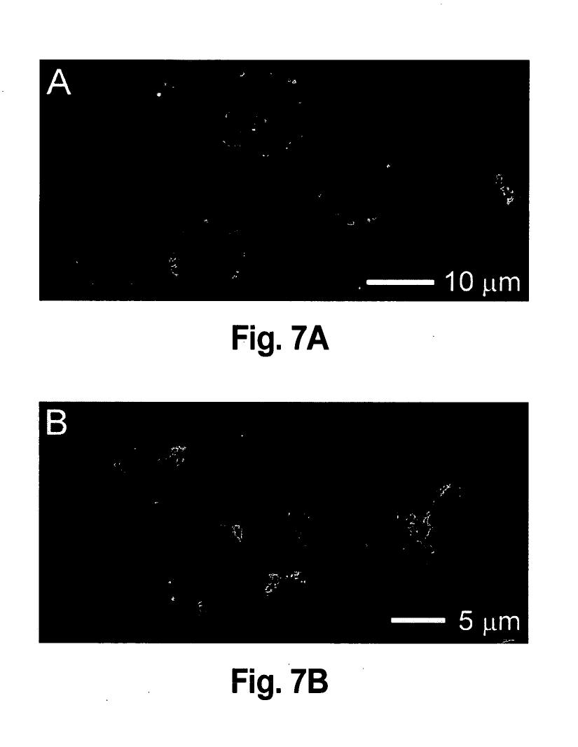

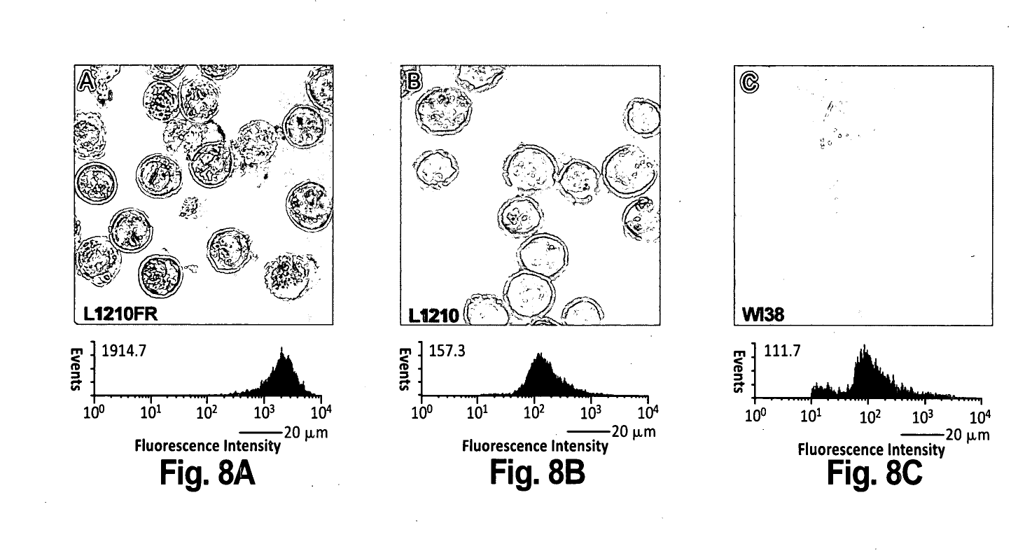

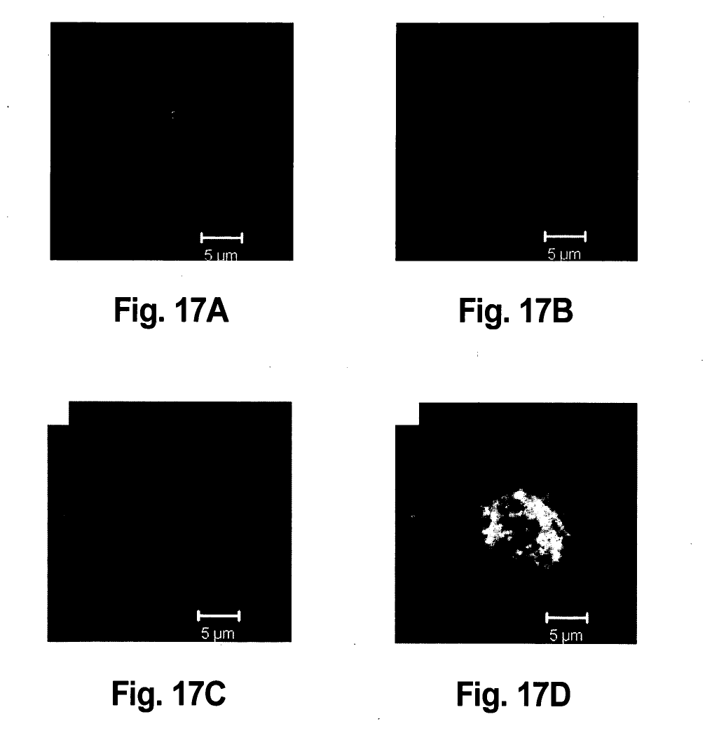

Building upon the promising results with biotin-SWNT conjugate 2 as a potentially versatile vehicle for tumor-targeting drug delivery, the efficacy of biotin-SWNT-linker-taxoid-fluorescein (3) was investigated for cellular uptake and drug release inside the leukemia. As the anticancer drug warhead, a highly potent 2^{nd}-generation taxoid, SB-T-1214, was used. The 2nd-generation taxoids exhibit 2-3 orders of magnitude higher potency against multidrug-resistant (MDR) cancer cell lines than Paclitaxel (Taxol®), which is the most widely used anticancer drug in the current chemotherapy. (Ojima et al., Bioorg. Med. Chem. Lett. 1998, 8, 189-194; Ojima et al., J. Med. Chem. 1996, 39, 3889-3896.) To evaluate the efficacy of the biotin-SWNT-taxoid conjugate 3 for its drug delivery and drug release, conjugate 3 was incubated with L1210FR cells at 50 μg/mL concentration for 3 h at 37° C. and washed the treated cells with PBS. As FIG. 8A shows, the internalization of conjugate 3 was confirmed by the bright fluorescence of the L1210FR cells observed by CFM. Next, the leukemia cells were treated with glutathione ethyl ester for an additional 2 h at 37° C. in order to secure the cleavage of the disulfide linkage covalently connecting the taxoid to the biotin-SWNT moiety. Then, the fluorescein-labeled taxoid released from the conjugate inside the leukemia cells should bind to tubulin/microtubule that is the target protein of the drug. In fact, as a CFM image in FIG. 8B clearly shows, the fluorescent taxoid did bind to the target protein to light up the large bundles of microtubules, which provides ultimate proof of the designed drug release. It should be noted that the intracellular glutathione in the leukemia cells should be able to cleave the disulfide linkage with much longer incubation time, but the endogenous glutathione level in cancer cells varies due to the significant difference in the physiological conditions between the cultivated cancer cells and those in the actual leukemia or solid tumors. Accordingly, the extracellular addition of excess glutathione ethyl ester is beneficial for a rapid visualization of the drug release inside the leukemia cells. This acceleration is evident by comparing FIG. 7A and FIG. 7B. To further confirm the microtubule network, the cells were incubated with conjugate 3 overnight, followed by removal of the excess conjugate 3, fixation of the cells, and staining with fluorescence-red labeled antibody. The overlay of the fluorescence green provided by the conjugate 3 and fluorescence red provided by the antibody (FIG. 17) can be clearly visualized.

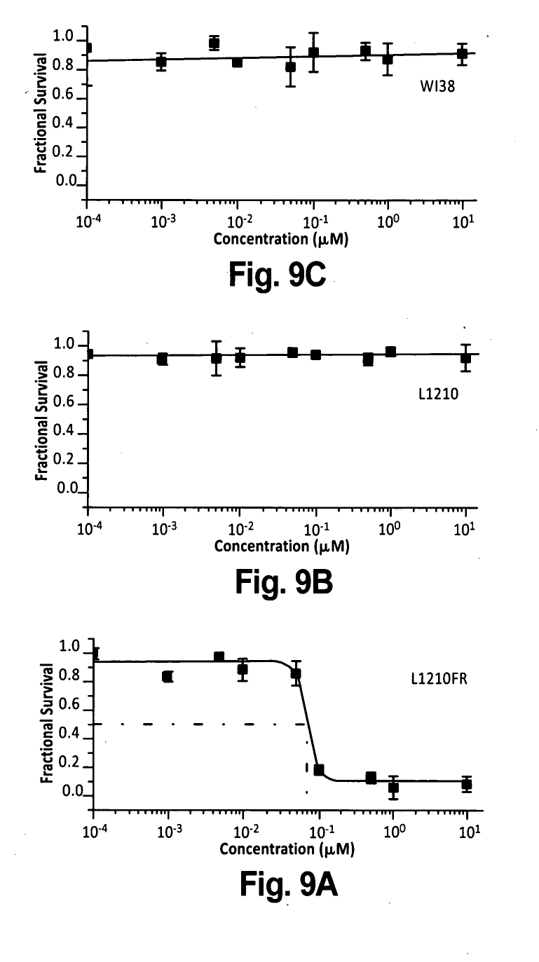

To show the specificity of the biotin-SWNT-taxoid-fluorescein (3) to cell lines that are over-expressed with the biotin receptor on their surface, two other cell lines were chosen, the mouse leukemia L1210 cell line and the W138 human embryonic fibroblast cell line, to compare with L1210FR cell line. Both the L1210 and W138 cell line are lack of the biotin receptor over-expressed on their surface. As a result, the conjugate 3 is expected to have much more cellular uptake to L1210FR as compared to the L1210 and W138 cells. FIG. 8 shows that the L1210FR cells (FIG. 8A) much stronger fluorescent intensity than the L1210 cells (FIG. 8B) and the W138 (FIG. 8C) upon the incubation with conjugate 3 at the same condition. The cytotoxicity of conjugate 3 was also compared to these three cell lines by the MTT assay. After 72 h incubation, the IC₅₀ value of the conjugate 3 to the L1210FR cell line is 0.36 μg/ml nanotubes, whereas the IC₅₀ value of cytotoxicity to both the L1210 and W138 cell lines are more than 50 μg/mL nanotubes, as shown in FIG. 9 and summarized in Table 1. In the control experiments, the IC₅₀ values of acid oxidized SWNTs, conjugate 1 (SWNT-FITC), and conjugate 2 (biotin-SWNT-FITC) to all three cell lines, are higher than 100 μg/mL. It is suggested that the cytotoxicity is only caused by the taxoid molecules that are released from the conjugate 3 (biotin-SWNT-taxoid-fluorescein).

According to calculations, the IC₅₀ value (0.36 μg/mL) of conjugate 3 corresponds to 51 nM of taxoid-fluorescein molecules in L1210FR cell line by assuming that all taxoid-fluororescein molecules attached to SWNTs are released. If the drug release is not complete, the IC₅₀ values should be even smaller (i.e., more potent). This means that the apparent cytotoxicity per taxoid is substantially increased by using the biotin-SWNT-based drug delivery system, i.e. 87.6 nM for drug itself (i.e., SB-T-1214-fluorescein) vs. 51 nM for conjugate 3. The results clearly indicate that the mass drug delivery into the cytosol of the cancer cells using this drug delivery system is superior than the simple exposure of the drug itself to the same cancer cells. The latter is very likely to include a concentration-dependent cell penetration efficiency factor (i.e., not all extracellular taxoids can be internalized). When taxoids get into the cancer cells through the mass drug delivery system, the released taxoids can quickly and tightly bind to the target protein (tubulins/microtubules) so that the effective intracellular drug concentration is substantially higher than that achieved by extracellular exposure of the drug.

TABLE 1
IC₅₀ values of the biotin-SWNT-taxoid conjugate
3 corresponding to different cell lines

| Cell Line | L1210FR | L1210 | WI38 |
|---|---|---|---|
| IC₅₀ (μg/mL) | 0.36 | >50 | >50 |

Thus, while there have been described what are presently believed to be the preferred embodiments of the present invention, those skilled in the art will realize that other and further embodiments can be made without departing from the spirit of the invention, and it is intended to include all such further modifications and changes as come within the true scope of the claims set forth herein.

1. A conjugate comprising a carbon nanotube with at least one covalently attached recognition module, and at least one covalently attached pharmaceutical compound or a precursor of the pharmaceutical compound,
wherein the pharmaceutical compound, or precursor of the pharmaceutical compound is attached to the carbon nanotube by a linker moiety.

2. A conjugate according to claim 1 wherein the recognition module is primarily attached to an end of the carbon nanotube, and wherein the pharmaceutical compound, or a precursor of the pharmaceutical compound, is primarily attached to the sidewall of the carbon nanotube.

3. A conjugate according to claim 1 wherein the recognition module is a protein, peptide, a polyunsaturated fatty acid, a saccharide, a lectin, an aptamer, a glycosaminoglycan, or a vitamin.

4. A conjugate according to claim 3 wherein the recognition module is biotin.

5. A conjugate according to claim 3 wherein the recognition module is folic acid.

6. A conjugate according to claim 3 wherein the recognition module is a monoclonal antibody.

7. A conjugate according to claim 3 wherein the recognition module is hyaluronic acid.

8. A conjugate according to claim 1 wherein the linker moiety is a disulfide-containing linker.

9. A conjugate according to claim 1 wherein the pharmaceutical compound, or the precursor thereof, is an antitumor drug, an antiangiogenic drug, a multi-drug reversal agent, an anti-inflammatory drug, an antibiotic, an antibacterial agent, an antiparasitic drug or an analgesic.

10. A conjugate according to claim 9 wherein the pharmaceutical compound is paclitaxel or a taxoid.

11. A conjugate according to claim 10 wherein the taxoid is docetaxel, SB-T-101131, SB-T-1102, SB-T-1103, SB-T-11033, SB-T-1104, SB-T-1212, SB-T-1213, SB-T-121303, SB-T-1214, SB-T-1216, SB-T-1217, SB-T-12851, SB-T-12852, SB-T-12853, SB-T-12854 or SB-T-1250.

12. A method of making a conjugate comprising a carbon nanotube with at least one covalently attached recognition module, and at least one covalently attached pharmaceutical compound or a precursor of a pharmaceutical compound, the method comprising:
(a) consecutively contacting a plurality of oxidized carbon nanotubes with (i) a first bifunctional amine having a first protecting group and (ii) a second bifunctional amine having a second protecting group to yield a plurality of carbon nanotubes with the first bifunctional amine primarily attached to the sidewall and the second bifunctional amine primarily attached to the end/detect sites; and
(b) replacing the first protecting group with a linker attached to the pharmaceutical compound or the precursor of a pharmaceutical compound; and
(c) replacing the second protecting group with the recognition module, wherein steps (b) and (c) occur consecutively and in either order to yield the conjugate.

13. The method of claim 12 wherein the first bifunctional amine is attached to the sidewall by 1,3-dipolar cycloaddition of azomethine ylide generated by condensation of an amino acid and an aldehyde.

14. The method of claim 12 wherein the first bifunctional amine is attached to the sidewall by [2+1]cycloaddition of nitrenes.

15. The method of claim 12 wherein the second bifunctional amine is attached to the end/defect sites of the nanotubes by reacting the nanotubes with N-(4-aminobutyl)-phthalimide.

16. The method of claim 12 wherein the second bifunctional amine is attached to the end/defect sites of the nanotubes by reacting the nanotubes with a 9-fluorenylmethyloxycarbonyl (FMOC) group.

17. The method of claim 12 wherein the protecting group is selected from the group consisting of a carbobenzyloxy group; a tert-butyloxy-carbonyl group; a 9-fluorenylmethyloxycarbonyl group; a benzyl group; and a p-methoxyphenyl group.

18. A method of claim 12 wherein the first bifunctional amine is N-(2-N-protecting group-ethyl)glycine.
